(19) United States
(12) Patent Application Publication
(10) Pub. No.: US 2019/0177679 A1
(43) Pub. Date: Jun. 13, 2019

(54) DEVICE FOR MANUFACTURE OF T-CELLS FOR AUTOLOGOUS CELL THERAPY

(71) Applicant: NDSU RESEARCH FOUNDATION, Fargo, ND (US)

(72) Inventors: Dharmakeerthi NAWARTHNA, Fargo, ND (US); Logeeshan VELMANICKAM, Fargo, ND (US); Vidura JAYASOORIYA, Fargo, ND (US)

(21) Appl. No.: 16/326,812

(22) PCT Filed: Sep. 7, 2017
(86) PCT No.: PCT/US2017/050493

§ 371 Date: Feb. 20, 2019

Related U.S. Application Data
(60) Provisional application No. 62/384,809, filed on Sep. 8, 2016.

(51) Int. Cl. C12M 1/00; B01J 19/08; C12M 1/42; C12N 5/0783

(52) U.S. Cl. CPC C12M 23/34; B01J 19/087; C12M 35/02; C12N 2501/515; C12N 2510/00; C12N 2501/599; C12N 5/0636

(57) ABSTRACT

The present invention relates to a new device for a scalable, biomanufacturing platform for the production of CAR-modified T-cells while eliminating on-target/off-tumor toxicity and decreasing the current production cost by 500 times (per treatment). The invention relates to a device to produce modified T-cells comprising a first chamber for proliferating a population of T-cells and a second chamber for modifying the T-cells to express a desired T-cell receptor antigen.

### RELATED APPLICATIONS

This application claims the benefit of U.S. Provisional Application Ser. No. 62/384,809, filed Sep. 8, 2016, the entire disclosure of which is incorporated herein by this reference.

### TECHNICAL FIELD

The presently-disclosed subject matter generally relates to devices and methods for the manufacture of T-cells. In particular, certain embodiments of the presently-disclosed subject matter relate to devices and methods for the manufacture of T-cells for autologous cell therapy.

### BACKGROUND OF THE INVENTION

Cell-based therapies, especially Chimeric Antigen Receptors (CAR) T-cell therapy utilizing engineered T-cells expressing CARs, have shown a great potential to revolutionize the treatments for malignant tumors. As illustrated in FIG. 1, the current process includes collecting T-cells from a patient; multiplying the T-cells; reengineering the T-cells; and infusing a patient with the reengineered T-cells. More specifically, T-cells are collected from whole blood samples taken from a patient or via apheresis, a process that withdraws blood from the body and removes one or more blood components (such as plasma, platelets, or white blood cells). The remaining blood is then returned back into the body and the collected T-cells are multiplied or “expanded” by growing the cells in a laboratory to increase the number thereof.

After multiplying the T-cells, they are sent to a laboratory or a drug manufacturing facility where they are genetically engineered to produce chimeric antigen receptors (CARs) on their surface. This can be by viral transfection or by inserting relevant mRNA into the cells. Following this reengineering, the T-cells are known as “chimeric antigen receptor (CAR) T-cells.” CARs are proteins that allow the T-cells to recognize an antigen on targeted tumor cells. These CAR T cells are frozen and, when there are enough of them, they are sent to the hospital or center where the patient is being treated.

Some practitioners of CAR T-cell therapy vary the order of the multiplying and reengineering steps, particularly in view of whether the transfection occurs by viral transfection. For example, if expression of the desired CAR molecules is by insertion of mRNA into the cells, expansion of the cells after inserting mRNA into the T-cells would essentially dilute out the mRNA since it does not replicate. Therefore, some T-cells would not be able to express CAR molecules.

Regardless of the order in which the multiplying and reengineering steps are performed, once at the hospital or treatment center the CAR T cells are infused into the patient. Many patients are given a brief course of one or more chemotherapy agents before they receive the infusion of CAR T cells. CAR T cells that have been returned to the patient's bloodstream multiply in number. These are the “attacker” cells that recognize, and kill, cancerous cells that have the targeted antigen on their surface. As the CAR T cells may remain in the body long after the infusion has been completed, they also guard against recurrence. Accordingly, the therapy frequently results in long-term remissions.

However, many obstacles still remain for CAR T-cell therapy. For example, although extensive research efforts have been continuing to develop CAR molecules targeting various tumors and subsequent analysis of efficacy, there has been no focus on developing manufacturing strategies that enable the production of CAR T-cells for therapy. For this reason, current production cost of a batch of CAR-modified T-cells for a single treatment is about $25,000. With therapies requiring up to 8-10 treatments, the total cost is typically at least about $200 k. Therefore, not only is the current process time consuming, it is expensive, and not widely available.

Additionally, the engineering of CAR modified T-cells is currently performed in a few centralized facilities using rudimentary biomanufacturing techniques, which result in heterogeneous mixtures of engineered CAR T-cells. These T-cells interact with nonpathogenic tissues and develop on-target/off tumor toxicity causing organ failures. As such, the current inability to manufacture homogenous mixtures of CAR expressing T-cells represents another significant hurdle to reengineered T-cell therapy. Thus, although CAR-based adoptive immunotherapy has the potential to revolutionize cancer treatments, these critical issues are hindering the translation of the latest developments in CAR T-cell biology into therapy.

One promising alternate approach to engineering T-cells with CAR receptors is through the transfection of T-cells with CAR producing mRNA using electroporation. Electroporation has been utilized to produce T-cells for cancer therapy targeting ErbB2 (Her2/neu) and CEA receptors in breast, pancreatic, lung and gastric cancers. In comparison with traditional viral-based transduction techniques, electroporation transiently expresses immunoreceptors (CAR) and therefore electroporation does not involve cellular genomic alteration, thereby eliminating adverse auto-aggression effects. However, this approach has not been previously achieved reliably, efficiently homogenously, or inexpensively.

This is due, in part, to the rudimentary nature of current two-electrode based electroporation techniques. As a result, controlled transfection of T-cells with mRNA molecules is not currently possible. This leads to the production of phenotypically different heterogeneous mixture of CAR modified T-cells causing these T-cells produce on-target/off tumor toxicity. This is creating a number of complex health issues leading to unnecessary consequences. Furthermore, the efficiency of the current T-cell electroporation is about 40%. The elimination of 60% of non-viable T-cells prior to treatments to avoid cross-reactions with other T-cells is technologically challenging because there are no distinct T-cell markers available for separation. These limitations in the current CAR modified T-cell production seriously undermine the true potential of adoptive immunotherapy.

Accordingly, there remains an urgent need for a viable engineering approach to cost-effectively produce CAR modified T-cells while minimizing the toxicity.

### SUMMARY OF THE INVENTION

In some embodiments, the presently-disclosed subject matter relates to a device to produce modified T-cells comprising a first chamber for proliferating a population of T-cells and a second chamber for modifying the T-cells to express a desired T-cell receptor antigen.

In one embodiment, chamber 1 and chamber 2 comprise a top surface, a bottom surface and four sides, and two of the sides are essentially parallel to each other and are the ends and another two sides are essentially parallel and are the sides and the top and bottom surface are parallel to each other and are at a distance of about 100 μm from each other.

In one embodiment, the top and the side surfaces are made of PDMS and the bottom surface is made of glass. In another embodiment, chamber 1 includes a series of T-traps and chamber 2 includes one or more channels bordered on each side by a metal, wherein the metal on one side is connected to one electrical pole and the other side is connected to a second electrical pole. Metals may be selected from the group consisting of gold (Au), silver (Ag), platinum (Pt), copper (Cu), iron (Fe), aluminum (Al), Tin (Sn), Nickel (Ni), Carbon (C), doped silicon (Si) or an alloy of metals.

In some embodiments, the electrical current for chamber 1 is about 100 mV and for chamber 2 is about 1.5V/P, where the pulse is about 1 ms to about 10 ms to achieve electroporation.

Also provided herein, in some embodiments, is a kit comprising the device disclosed herein combined with one or more of the accessories selected from the group consisting of syringes, nutrients, food, cytokines, buffers, storage containers, transfer vehicles and instructions

### DESCRIPTION OF THE FIGURES

FIG. 1 shows the present method for performing CAR T-cell Therapy.

FIG. 2 shows the basic steps of the present invention for manufacturing T-cells with CAR receptors.

FIG. 3 shows Schematic representation of the scaled-up, integrated T-cell manufacturing device to produce 10⁷ T-cells.

FIGS. 4A-C show cells and beads trapped using DEP. (A) Combined bright field and fluorescent image of the electrodes and trapped fluorescent beads (diameter=10 micrometers) using negative DEP. Note that clusters of beads are trapped. (B) Bright field view of trapped C2C12 cells using negative DEP (white rectangles with broken lines). Clusters of cells were trapped near the electrodes. (C) Fluorescent image of the trapped C2C12 cells using positive DEP. Note that single C2C12 cells (white circles with broken lines) are trapped on the electrodes. Scale bars indicate 100 micrometers.

FIGS. 5A-C show schematic diagram of the proposed T-cell engineering for expansion; (A, B, C) T-cell expansion. (A) Trap T-cells using DEP, (B) Flow antibody coated beads, (C) expansion of T-cells.

FIGS. 6A-C show the design and fabrication of DEP traps for T-cells. (A) Picture of a micro electrode array with 20,000 single cell traps. Electrodes were fabricated in Au on glass substrate. Inset shows the magnified view of a DEP trap. (B) The calculated electric field distribution around electrodes. When T-cells trap using positive DEP, they will be trapped on the electrodes as single cells in A. When negative DEP is used, T-cells will be trapped in B. (C) Calculated electric field gradients around the electrodes. These electric field gradients generate high DEP forces on T-cells resulting in high-throughput trapping.

FIGS. 7A-B show electroporation of protein calcein A. (A) Patterned cells separated outside the chamber following electroporation with calcein A and green dye. (B) Patterned cells separated outside the chamber following electroporation with calcein A and red dye.

FIGS. 8A-B show images illustrating viability of cells after electroporation. (A) Growth of electroporated cells after 24 hours (left) as compared to cells after electroporation (right). (B) Growth of electroporated cells after 48 hours (left) as compared to cells after electroporation (right). All images shown at 10× zoom.

FIGS. 9A-B show images illustrating how the cells retain the protein after electroporation. (A) 10× zoom image of protein remaining in the cells after 48 hours. (B) 25× zoom image of protein remaining in the cells after 48 hours.

FIGS. 10A-C shows schematic diagram of the proposed T-cell engineering for electroporation. (A) flow T-cells over IDEs, (B) T-cell patterning using DEP, (C) massively parallel electroporation of single T-cells. (A and B) T-cell purification and (A-C) transfection of T-cells with mRNA.

FIGS. 11A-C show the designed and fabricated IDE array for T-cell pattering and electroporation. (A) Picture of the fabricated IDE array and inset illustrate the close-up view of the individual IDE pairs. (B) Demonstration of the particle patterning using DEP. Polystyrene beads (diameter=10 μm) were trapped and patterned between individual IDEs using DEP. Rectangle with white broken lines shows perfectly aligned beads. (C) Patterning of fibroblast cells (CRL—1764) using DEP. Scale bars indicate 30 μm.

FIGS. 12A-C shows quantitative injection of mRNA molecules using electroporation. (A) Time dependent mRNA transport into T-cells. Scale bars indicate 5 μm. (B) correlation between electroporation time and number of mRNA transport into single T-cell. (C) comparison of proposed electroporation and traditional electroporation.

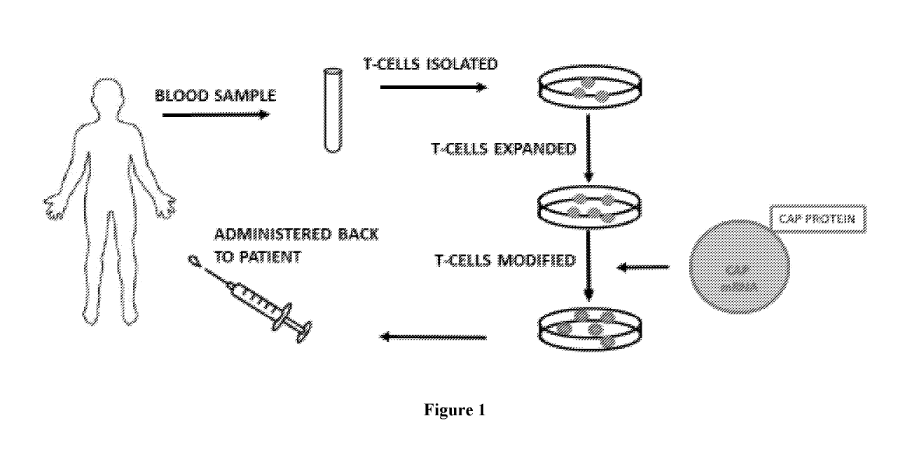

### DETAILED DESCRIPTION OF THE INVENTION

As used herein, the terms “first chamber,” “chamber 1,” and “region 1” may all be used interchangeably.

As used herein, the terms “second chamber,” “chamber 2,” and “region 2” may all be used interchangeably.

The term “DEP,” as used herein, means dielectrophoresis or the migration of particles toward the position of maximum field strength or electric field gradient in a nonuniform electric field.

The term “IDE,” as used herein, means micro-interdigitated electrodes.

As used herein, the terms “protein,” “protein ligand,” and “ligand” may all be used interchangeably.

The term “PDMS,” as used herein, means polydimethylsiloxane.

The term “rms,” as used herein, means root mean square.

The presently-disclosed subject matter relates to a new device for a scalable, biomanufacturing platform for the production of CAR-modified T-cells while eliminating on-target/off-tumor toxicity and decreasing the current production cost by 500 times (per treatment). In some embodiments, the presently-disclosed subject matter includes a device to produce modified T-cells comprising a first chamber for proliferating a population of T-cells and a second chamber for modifying the T-cells to express a desired T-cell receptor antigen.

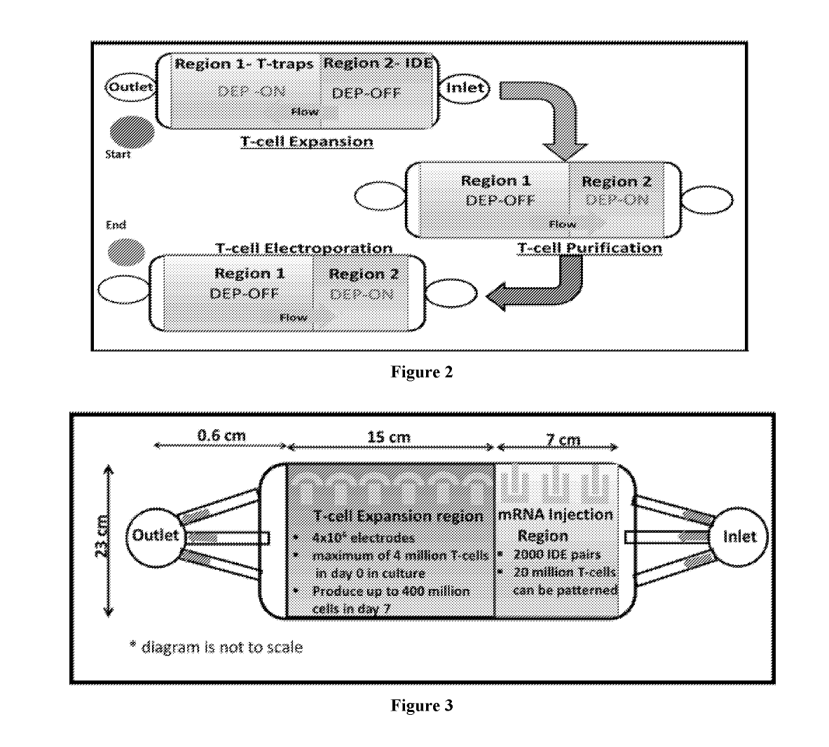

In some embodiments, the device includes a first chamber and a second chamber. Each of chamber 1 and chamber 2 comprises two parallel surfaces and four sides. Two of the sides are parallel to each other and are the side sides. The other two sides are somewhat parallel to each other and are the ends. One or more of the two end sides may be curved or beveled to improve the efficiency of the flow in and out of the chambers. The parallel surfaces are the top and bottom of the chambers and are usually rectangular-like in shape, except one or more of the edges of the top and bottom surfaces may be curved or beveled to match one or more of the curved or beveled sides. The top and bottom are separated from each other by a distance of from about 30 μm to about 1000 μm, preferably by a distance of from about 30 μm to about 400 μm, more preferably by a distance of from about 30 μm to about 200 μm, most preferably by a distance of about 100 μm. The two end sides of each chamber that are parallel to each other may have openings or ports to allow T-cells to flow into and out of the chambers. The top and sides of the chamber 1 can be any suitable material, including, but not limited to, glass, PDMS, or a combination thereof. For example, in one embodiment, the bottom material is glass and the top and side material is PDMS. In a preferred embodiment of the invention, one end side of chamber 1 and one end side of chamber 2, usually end sides that are not curved or beveled, can abut each other so that the two chambers are one integrated unit sharing one end of each chamber. FIG. 2 and FIG. 3 illustrate the top view of a preferred integrated device chamber comprising both chamber 1 and chamber 2.

The dimensions of chamber 1 can be from about 10 cm to about 50 cm end to end, preferably from about 10 cm to about 30 cm end to end, more preferably from about 10 cm to about 20 cm end to end. The dimensions of chamber 1 can be from about 10 cm to about 75 cm side to side, preferably from about 10 cm to about 50 cm side to side, more preferably from about 20 cm to about 30 cm side to side.

In one embodiment, chamber 1 is about 15 cm long on its side sides, about 23 cm wide on its end sides, and about 100 μm deep for the width of the sides and ends. In another embodiment, chamber 2 is about 7 cm long on its side sides, about 23 cm wide on its end sides, and about 100 μm deep for the width of the sides and ends.

A suitable population of T-cells to be proliferated in the first chamber (also known as chamber 1) can be obtained from a blood sample. The blood sample can be any blood sample but preferably is obtained from the patient to be treated with the CAR modified T-cells. Purification and isolation of T-cells from the blood sample can be accomplished by known methods. The T-cell population is suspended in a suitable buffer for insertion into chamber 1. The starting T-cell population is from about 1 million cells to about 50 million cells, preferably from about 5 million cells to about 25 million cells, most preferably about 10 million cells. Typically, 5 ml of human blood contains about 2.5-9.0 million T-cells.

The role of chamber 1 is to expand or proliferate a population of T-cells to significantly increase their number. A significant increase is from about 100 fold to a maximum of about 1000 fold. This is accomplished by trapping small subpopulations of the T-cells to increase their density. With a higher density of T-cells in a localized area, they can be subjected to known reagents and cytokines to increase their number. Trapping the T-cells occurs using cell traps, micro-traps or T-traps that have a particular geometrical shape, and also applying an electric field to the cells in a T-trap. The T-traps are individually a localized structure, but they do have access to the entire area of chamber 1 so that cells can flow into and out of the T-traps under appropriate conditions.

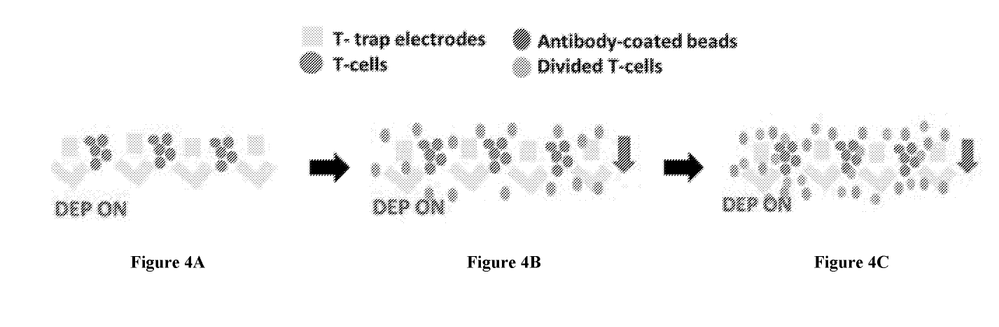

In some embodiments, the T-traps are fabricated to be within the enclosed chamber 1. In one embodiment, each trap includes a capture container and a guiding container. In another embodiment, each container is constructed of an electrical conducting material and attached to a potential source of electric current. In some embodiments, the containers are constructed of a metal material such as gold (Au), silver (Ag), platinum (Pt), copper (Cu), iron (Fe), aluminum (Al), Tin (Sn), Nickel (Ni), Carbon (C), doped silicon (Si) or an alloy of metals. Preferred electrical conducting materials are Au, Ag, Pt and indium tin oxide. The capture container can be a flat surface or a concave surface with the opening of the concave surface facing the guiding container. FIGS. 3, 4A-C, and 5A-C show preferred configurations of the two containers. In this way, the starting population of T-cells can be electrically guided to the capture container to increase a localized subpopulation of T-cells for expansion. As long as the electric potential is maintained the T-cell population stays concentrated at or near the capture container. The electric potential that is used to guide the T-cells is from about 10 milli Volts (mV) to about 200 mV, preferably at about 100 mV.

To allow for expansion of the T-cell population, the open, unoccupied volume of chamber 1 should be sufficient to allow at least 100 million, preferably at least 500 million, and more preferably at least a billion T-cells after expansion takes place. T-cells typically have a diameter of about 20-40 μm. To encourage growth, the media in which the T-cells are captured can contain reagents selected from the group consisting of nutrients, food and cytokines. During the preferred 1000 fold expansion, components to produce cellular structure, energy production and growth will shorten the time for the expansion to take place. The appropriate nutrients, food and cytokines for a particular population of T-cells is known to those of skill in the art as is the temperature and other growth conditions. Growth and expansion of the T-cell population can be monitored by standard techniques such as spectrophotometry and standard curves.

Once the T-cell population in chamber 1 reach the desired expanded cell population amount and/or density, the electrical field can be turned off so the T-cell population in chamber 1 can be moved to chamber 2 by physically flushing the cells. In certain embodiments, where chamber 1 and chamber 2 abut each other, inserting a syringe into an opening of the side opposite the side of chamber 1 that abuts chamber 2 will allow the T-cells to be flushed through and opening in the shared side between chamber 1 and chamber 2.

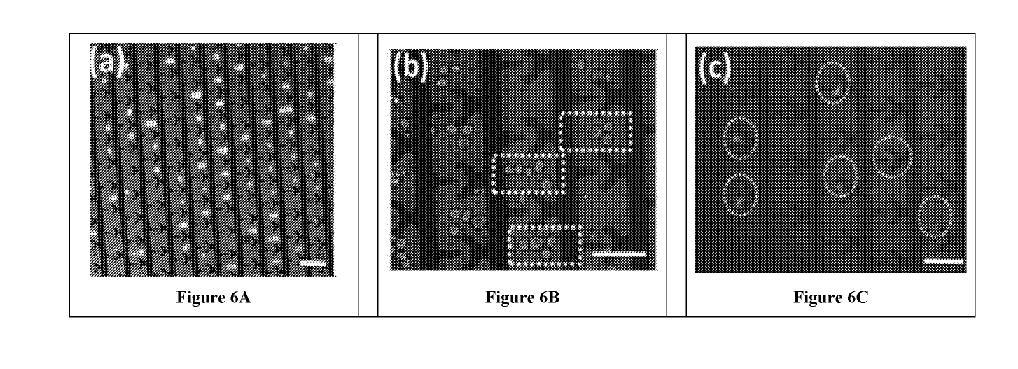

In some embodiments, during T-cell expansion, negative DEP is used to form T-cell clusters as indicated in FIG. 6B and initiate T-cell expansion. This facilitates close monitoring of the T-cell expansion rate. Alternatively, if there is not acceptable expansion (˜100×), positive DEP (with single T-cells) may be used to expand the T-cells. If positive DEP is effectively expanding T-cells, static conditions may be used to expand T-cells. Briefly, T-cells are trapped using −ve or +DEP and turn off the traps and culture cells with beads. Negative DEP is a repulsion force on T-cells from the electrodes. In negative DEP, T-cells will move away from the electrode and go to the weakest electric field area. The positive DEP is the exactly the opposite of the negative DEP. In one embodiment, the T-cell expansion is about 100×. In another embodiment, increasing T-cell expansion includes increasing the number of T-traps and/or increasing the number of T-cells in day 0. In a further embodiment, if there is not rapid expansion within 10 days, a flow of a mixture of Interleukin-2 (IL-2, 10³ IU/mL), anti-CD3 (0.1 μg/mL) and anti-CD28 (0.25 μg/mL) may be applied. Without wishing to be bound by theory, it is believed that this mixture will efficiently expand T-cells.

The role of chamber 2 is to modify the T-cells in the expanded T-cell population so that they will present on their surface membrane a protein, protein ligand or ligand, which will bind to one or more cell surface proteins on a targeted cancer cells. In one embodiment, the protein ligand is chimeric in that besides a region that will bind to one or more cell surface proteins on targeted cells, such as cancer cells, it also has a region that that includes hydrophobic amino acids so that it will bind to the T-cell membrane through hydrophobic bonding. As a result, in another embodiment, the protein ligand is presented outside the T-cell and is available to bind cell surface proteins on the targeted cells. In a further embodiment, this brings the T-cell in contact with the targeted cells, permitting the T-cell to perform the role of a T-cell and kill targeted cancer cells. In this way, the modified T-cells will bind specifically to the targeted cells.

Once the T-cells bind, they can perform the role of a T-cell and kill the targeted cell, such as a cancer cell. The most effective way to do this is to insert genetic material into most, if not all, the T-cells of the expanded T-cell population so the T-cells will express the protein coded by the mRNA. A number of such mRNA molecules are known that code for such chimeric antigen receptor (CAR) proteins that bind to various cancer cells. However, the device of the present invention allows a more homogenous insertion of the mRNA into the T-cells in a much shorter time and at a much lower cost. By homogenous insertion, it is meant each T-cells received a similar number of mRNA molecules and most, if not all, T-cells receive that number of mRNA molecules. A preferred amount of mRNA used per T-cell sample, after expansion (up to about a billion T-cells) is from about 100 μg/mL to about 1000 μg/mL, preferably about 100 μg to about 700 μg/mL, more preferably about 200 μg/mL to about 500 μg/mL. The mRNA is preferably in a homogenous distribution in the solution in chamber 2 before, during and after the patterning of the T-cells occurs.

The magnitude of induced electric potential (electric field) in T-cell membranes determines the size of the pore that is used to transport exogenous mRNA molecules through electroporation. Therefore, in some embodiments, producing a homogenous mixture of T-cells transfected with mRNA molecules includes inducing a uniform electric field in all T-cell membranes in the cell mixture. In one embodiment, inducing a uniform electric field includes patterning T-cells in single files in a channel between parallel micro-electrodes so that every single T-cell will be subjected to the same external electric field. In another embodiment, the micro-electrodes include micro-interdigitated electrodes (IDE). Lithography based microfabrication can be used to fabricate the IDE in chamber 2 of the present invention using any of the electrical conducting materials used in chamber 1 on glass substrates. The space between electrodes can be anywhere from about the diameter of typical T-cells to about twice the diameter of typical T-cells, or preferably about 30 μm to accommodate a single file of T-cells between electrodes.

With one or more channels for T-cell patterning constructed in chamber 2, T-cell patterning is achieved through the use of DEP. The IDE/DEP patterning of the T-cells can use an AC electric potential of from about 20 mV to about 500 mV, preferably from about 60 mV to about 200 my, most preferably at about 100 mV. FIG. 11A illustrates the IDEs of one preferred embodiment designed and fabricated for cell patterning/electroporation experiments. Under these electric field conditions, more than 99% of the T-cells that were on the electrodes can be patterned. Finally, we turned off the electric field (DEP) and observed that patterned T-cells do not move or become disturbed.

The dimensions of chamber 2 can be from about 4 cm to about 25 cm end to end, preferably from about 5 cm to about 20 cm end to end, more preferably from about 5 cm to about 10 cm end to end. In one embodiment, for example, the dimensions of chamber 2 is about 7 cm long on its side sides, about 23 cm wide on its end sides and about 100 μm deep for the width of the sides and ends. The dimensions of chamber 1 can be from about 10 cm to about 75 cm side to side, preferably from about 10 cm to about 50 cm side to side, more preferably from about 20 cm to about 30 cm side to side.

Once the T-cells are patterned into a single file, electroporation can be performed. This is achieved by applying a DC current pulse (DCP) of a suitable strength and for a suitable time across the electrodes lining each side of the T-cell patterning channel. For example, in some embodiments, A DCP of suitable strength and duration to electroporate T-cells includes any DCP that provides about 1.25×10⁵ V/m induced electric field in the T-cell membrane. Suitable DCP current strength can be from about 0.1×10⁵V per pulse (V/P) to about 10×10⁵V/P, preferably from about 0.5×10⁵V/P to about 4×10⁵V/P, most preferably about 1.5V/P. The DCP current can have a pulse (P) length for a period of time from about 0.1 ms (milli seconds) to about 50 ms, preferably from about 1 ms to about 10 ms, post preferably about 5 ms. Electroporation equipment is commercially available.

Following electroporation, the T-cells can be removed from chamber 2, suspended in an appropriate buffer and the T-cells purified from other components in chamber 2 such as medium, proteins, nutrients, cytokines, etc. and then stored for up to about 10 days before further formulation and administration to a patient in need thereof. In some embodiments, for example, the device disclosed herein produces modified T-cells for treating cancer. The modified T-cell population may be formulated and administered to a patient as determined by the attending physician. Administration of the T-cells includes any suitable methods, such as, but not limited to, injection or infusion over a period of time. The device produces sufficient modified T-cells for at least a single administration, although more than one cycle of administration may be called for depending on the stage of the cancer, age, and health of the patient and the like.

Accordingly, the instant disclosure provides a device that is inexpensive, rapid, easy to manufacture, easy to use, efficient, effective, and capable of producing CAR T-cells for administration to patients where the patient is located. In addition, as compared to existing devices and methods, the device disclosed herein provides markedly improved CAR modified T-cell production while reducing or eliminating the toxicity and high production cost. By overcoming on-target/off-tumor toxicity and high production costs, the presently-disclosed subject matter provides an innovative solution that permits widespread adoption and may be the most transformative development in cancer therapy since the discovery of chemotherapeutics.

Although discussed above with regard cancer patients and cancer therapy, as will be appreciated by those skilled in the art, the instant disclosure is not so limited. Other disease to which the instant disclosure is applicable include, but are not limited to, infections, heart diseases, and stem cell therapy, all of which are expressly contemplated herein.

In addition, although discussed above with regard to T-cells, as will be understood by those skilled in the art, the presently-disclosed subject matter it not so limited and may include any other field or type of cell where electroporation is desired. For example, in one embodiment, the device disclosed herein may be used for stem cell research and/or stem cell electroporation. In another embodiment, the presently-disclosed subject matter reduces or eliminates the lysing of stem cells as compared to existing electroporation techniques, where 99% of stem cells are lysed during the transfection of genes/proteins.

Also provided herein is a kit including the device according to one or more of the embodiments disclosed herein combined with compatible accessories to make use of the device easier. Such accessories can include one or more items selected from the group consisting of syringes, nutrients, food, cytokines, buffers, storage containers, transfer vehicles and the like. The kit can also include instructions for optimal use of the device.

The details of one or more embodiments of the presently-disclosed subject matter are set forth in this document. Modifications to embodiments described in this document, and other embodiments, will be evident to those of ordinary skill in the art after a study of the information provided in this document. The information provided in this document, and particularly the specific details of the described exemplary embodiments, is provided primarily for clearness of understanding and no unnecessary limitations are to be understood therefrom. In case of conflict, the specification of this document, including definitions, will control.

When the term “including” or ‘including, but not limited to” is used, there may be other non-enumerated members of a list that would be suitable for the making, using or sale of any embodiment of this invention.

While the terms used herein are believed to be well understood by those of ordinary skill in the art, certain definitions are set forth to facilitate explanation of the presently-disclosed subject matter.

Unless defined otherwise, all technical and scientific terms used herein have the same meaning as is commonly understood by one of skill in the art to which the invention(s) belong.

All patents, patent applications, published applications and publications, GenBank sequences, databases, websites and other published materials referred to throughout the entire disclosure herein, unless noted otherwise, are incorporated by reference in their entirety.

Although any methods, devices, and materials similar or equivalent to those described herein can be used in the practice or testing of the presently-disclosed subject matter, representative methods, devices, and materials are described herein.

The present application can “comprise” (open ended) or “consist essentially of” the components of the present invention as well as other ingredients or elements described herein. As used herein, “comprising” is open ended and means the elements recited, or their equivalent in structure or function, plus any other element or elements which are not recited. The terms “having” and “including” are also to be construed as open ended unless the context suggests otherwise.

Following long-standing patent law convention, the terms “a”, “an”, and “the” refer to “one or more” when used in this application, including the claims. Thus, for example, reference to “a cell” includes a plurality of such cells, and so forth.

Unless otherwise indicated, all numbers expressing quantities of ingredients, properties such as reaction conditions, and so forth used in the specification and claims are to be understood as being modified in all instances by the term “about”. Accordingly, unless indicated to the contrary, the numerical parameters set forth in this specification and claims are approximations that can vary depending upon the desired properties sought to be obtained by the presently-disclosed subject matter.

As used herein, the term “about,” when referring to a value or to an amount of mass, weight, time, volume, concentration or percentage is meant to encompass variations of in some embodiments ±20%, in some embodiments ±10%, in some embodiments ±5%, in some embodiments ±1%, in some embodiments ±0.5%, and in some embodiments ±0.1% from the specified amount, as such variations are appropriate to perform the disclosed method.

As used herein, ranges can be expressed as from “about” one particular value, and/or to “about” another particular value. It is also understood that there are a number of values disclosed herein, and that each value is also herein disclosed as “about” that particular value in addition to the value itself. For example, if the value “10” is disclosed, then “about 10” is also disclosed. It is also understood that each unit between two particular units are also disclosed. For example, if 10 and 15 are disclosed, then 11, 12, 13, and 14 are also disclosed.

As used herein, “optional” or “optionally” means that the subsequently described event or circumstance does or does not occur and that the description includes instances where said event or circumstance occurs and instances where it does not. For example, an optionally variant portion means that the portion is variant or non-variant.

### EXAMPLES

### Example 1: Integrate Basic Steps of Car Modified T-Cell Engineering and Demonstrate the Production of Car Modified T-Cells (˜10 Million) for a Single Treatment at 500 Times Cheaper than Current Cost

FIG. 2 illustrates the basic steps involved in manufacturing about 10 million T-cells expressing ErbB2 immunoreceptor molecules. Briefly, isolated T-cells are expanded in the T-trap electrode area (region 1), (T-cell expansion). In Examples 2 and 5 below, the experimental conditions needed for T-cell expansion are determined. The DEP in the T-cell expansion area is then turned off and cells are flown over IDE electrodes (region 2) when DEP is applied in the IDE area (T cell purification). T-cells are selectively patterned in the IDEs. Since beads do not experience DEP, this step purifies the T-cells by washing away beads. The experimental conditions from Example 2 are then used to trap T-cells, which is followed by flowing in the ErbB2 mRNA and electroporating T-cells (T-cell electroporation). Finally, electroporated T-cells are collected and suspended in the RPMI 1640 and incubated for the expression of immunoreceptors. Thereafter, the expression levels of ErbB2, cytotoxicity, and cytokine release during the presence of target tumor cells is analyzed. Based on the results, necessary adjustments to the design and critical experimental parameters are determined.

To integrate procedures, an IDE electrode array and a T-trap array were fabricated in a single device. These electrode arrays were fabricated in Au (gold) on a glass substrate. A simple layout of the scaled-up device is illustrated in FIG. 3. 12 inch commercially available glass wafers (University Wafer, Boston, Mass.) were used to fabricate a prototype of the device. Fabrication of electrodes included soft lithography, sputtering of 1000 Å layer of Au and traditional photoresist lift-off in Acetone. The detailed fabrication procedure is reported elsewhere.

Once the electrode manufacturing is finished, the glass wafer was diced to achieve the desired dimensions of the device. A Polydimethylsiloxane (PDMS) flow channel was then produced to flow T-cells in and out of the device. PDMS provides significantly improved temperature and gas exchange between proliferating T-cells and outside, resulting in favorable culture conditions inside the device. Briefly, a PDMS mold was manufactured to the desired dimensions using 3D printing and commercially available PDMS and was poured onto the mold and cured in an oven. After curing for 3-4 hours, PDMS channels were peeled off from the mold. Inlets and outlets were made using a commercially available hole punch. Finally, PDMS channels and Electrodes were exposed to O₂ plasma and bonded to produce the device.

The lytic and activating thresholds are used to find the minimum immunoreceptor level needed to produce T-cells. Once the immunoreceptor level is determined, the device expands, purifies, and electroporates T-cells, producing a number of T-cells sufficient for a single treatment. The overall objective is to integrate the basic steps into a single integrated device to produce about 10⁷ T-cells. The overall outcome of this is to demonstrate the production of ErbB2 modified T-cells for a single treatment cost-effectively.

Finally, the potency of the manufactured T-cells in eliminating tumors is monitored. The instant inventors have developed an electrical impedance based label-free method that detects the dielectric properties of the cells at single-cell resolution. The T-cells must be patterned in IDE electrodes to measure the dielectric properties. The process involves measuring the bulk impedance of the cell/medium and uses Scharama's impedance model to find the dielectric properties of individual T-cells. Therefore, the technology disclosed herein qualifies as one of the cGMP manufacturing technologies.

The resulting information is then used to develop a massively parallel single-cell electroporation technique to quantitatively transfect T-cells with CAR expressing mRNA molecules and produce homogenous mixture of CAR expressing T-cells. This capability is utilized to produce the minimum number of immunoreceptors needed in T-cells to efficiently lyse tumor cells thereby minimizing the interactions with healthy tissues and eliminating on-target/off-tumor toxicity. This T-cell manufacturing procedure is scaled-up through the integration of T-cell engineering, expansion and purification to produce a large number of CAR-modified T-cells. In each step, T-cells are manipulated through the use of dielectrophoresis (DEP), the phenomenon in which a force is generated selectively on T-cells when cells are exposed to non-uniform electric fields.

### Example 2: Isolation of T-Cells from Patient Blood Sample

Resting CD4⁺ and CD8⁺ T cells are isolated from whole human blood obtained from Bioreclamations Inc (Westbury, N.Y.) using a well-established technique. Briefly, lymphocytes are isolated using a density gradient by layering blood on top of the Ficoll-Paque followed by mononuclear removal and red blood cell lysis. Finally, resting CD4⁺ and CD8⁺ T cells are negatively isolated using standard Miltenyi isolation kits (Cambridge, Mass.). The isolated T-cells are suspended in RPMI 1640. Standard quality control procedures are implemented to check T-cells.

The same procedure can be used with patient blood.

### Example 3: Expansion of T-Cell Population in Chamber 1

As indicated in FIGS. 4A-C, during T-cell expansion, T-cells are trapped on the electrodes. FIG. 2 illustrates the basic steps of the production of CAR T-cells and FIGS. 4A-C show the steps of T-cells expansion, which involves trapping T-cells as clusters using negative DEP, flow CD3+/CD28+ beads and expansion.

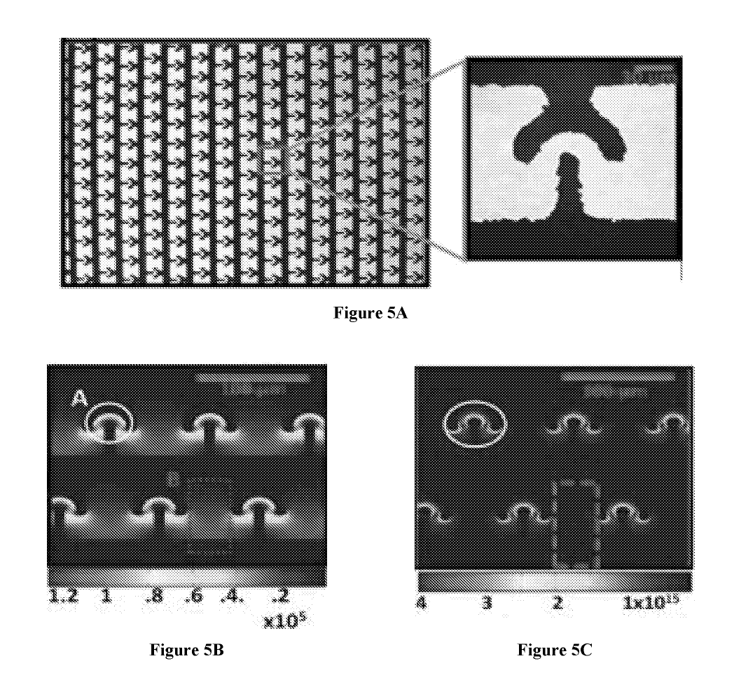

To successfully achieve this, a new class of electrodes that are capable of generating highly localized electric field gradients near electrodes to trap T-cells was designed (FIG. 5A). These electrodes are called T-traps, and a magnified view of a T-trap electrode is shown in FIG. 5A-inset. The instant inventors used the fabrication procedure utilized in fabricating IDEs. COMSOL software was then used to calculate the electric fields and electric field gradients that can be expected from T-traps. FIGS. 5B-C illustrate the calculated electric field (V/m) and field gradient (V²/m³) respectively. The electric fields and gradients highly localized between electrodes and T-traps are used to make T-cell patterns in well-defined locations. Spacing between patterns can be changed easily. There are two modes of patterning: T-cells can be patterned on the electrodes using positive DEP (white circle with A show the location of traps) or using negative DEP (red rectangle with B show the location).

In this Example, the device is utilized to study T-cell trapping. In particular, the external potential (peak to peak voltage required to generate electric field gradients), frequency, and conductivity of the buffer needed for trapping is determined. For each experimental condition, T-cells are trapped and free (non-trapping) T-cells are removed from the device. The DEP force is then turned off, the trapped cells are collected, and a cell count is performed. Using the T-cell counts, the percentage of trapped T-cells is calculated. The experimental condition that produces the maximum amount of trapping (that produces the maximum percentage-cells) is determined.

First, the conditions needed for expanding trapped T-cells by 100 fold are determined. T-cell expansion is a well-studied problem in the literature. A number of successful protocols have been developed to expand T-cells by 100-fold within about 10 days. In published studies, T-cells were expanded in static conditions where T-cells and other necessary reagents (antibodies) were placed in a stationary state in regular cell culture flasks/bioreactors. The instant inventors created a natural biological environment ex vivo in the device for T-cell expansion. By doing so there is faster expansion of T-cells. Briefly, using negative DEP, T-cells are placed as clusters with well-defined spacing. A flow of CD3⁺/CD28⁺ coated 5 μm diameter Dynabeads (Life technologies, Carlsbad, Calif.) is then applied around those clusters and the T-cell expansion is studied. The outlet of the device is designed to circulate or flow beads out of the device without flowing the expanding T-cells out of the device. The instant inventors studied the effects of the CD3+/CD28+ coated Dynabeads on the number of T-cells in clusters, the flow rate of the Dynabeads, and the ratio between T-cells and Dynabeads to achieve high-throughput expansion of T-cells. Finally, the optimum conditions for expanding T-cells in the instant device were determined, with these conditions believed to expand T-cells much faster than the static methods.

Next, the instant inventors performed experiments to study the DEP-based cell trapping. Commercially available fluorescent polystyrene beads (diameter=10 μm and green fluorescent protein on the surface) were patterned using negative DEP by applying a 10 kHz electric field. FIG. 6A illustrates the combined bright-field and fluorescent image showing locations of the traps with respect to T-trap electrodes. Note that clusters of beads are trapped near electrodes.

C2C12 cells were then suspended in 1×PBS buffer and flowed over the electrodes, an electric potential (10 Vpp and 200 kHz) was applied, and the cells were trapped on the electrodes. FIG. 6B shows a bright field image of trapped cells using negative DEP. The trapped cells are in the rectangle with white broken lines. In parallel, the T-cells were stained with Calcein AM (Thermo scientific) and trapped using positive DEP (10 Vpp and 50 kHz). FIG. 6C shows a fluorescent image of trapped cells. Trapped cells are inside the circles with white broken lines. Note that single cells are trapped using positive DEP and a cluster of cells is trapped with negative DEP. These results strongly support a more efficient method for expanding T-cells.

### Example 4: Verification and Viability of Electroporated Cells

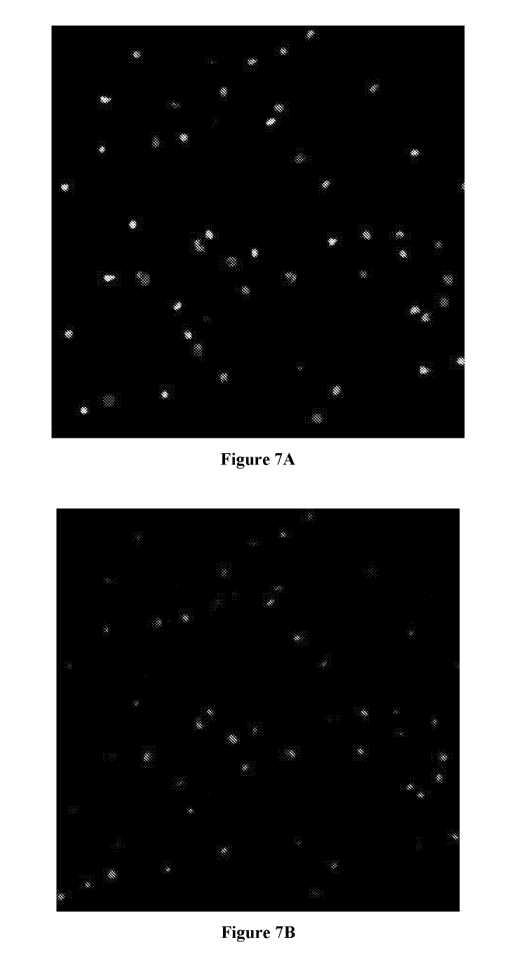

To verify electroporation, cultured fibroblast cells were patterned as discussed above and then electroporated with calcein A, which is a labeled protein having a size of about 4-5 mg. After electroporation, the cells were separated outside the chamber. As shown by the fluorescence in FIGS. 7A-B, the labeled calcein A was electroporated into the cells. Although demonstrated with calcein A, these results are believed to be representative of electroporation with mRNA according to the instant disclosure.

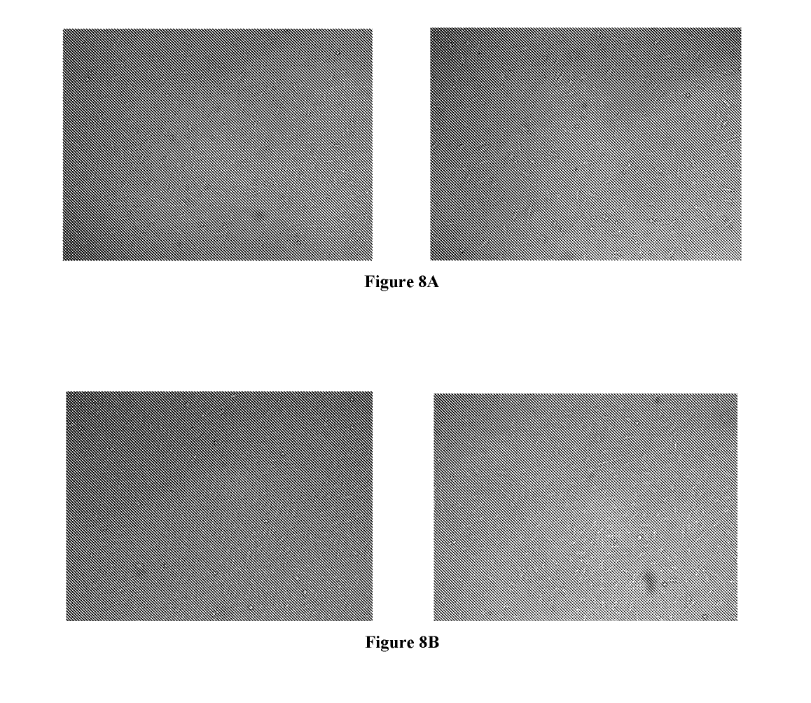

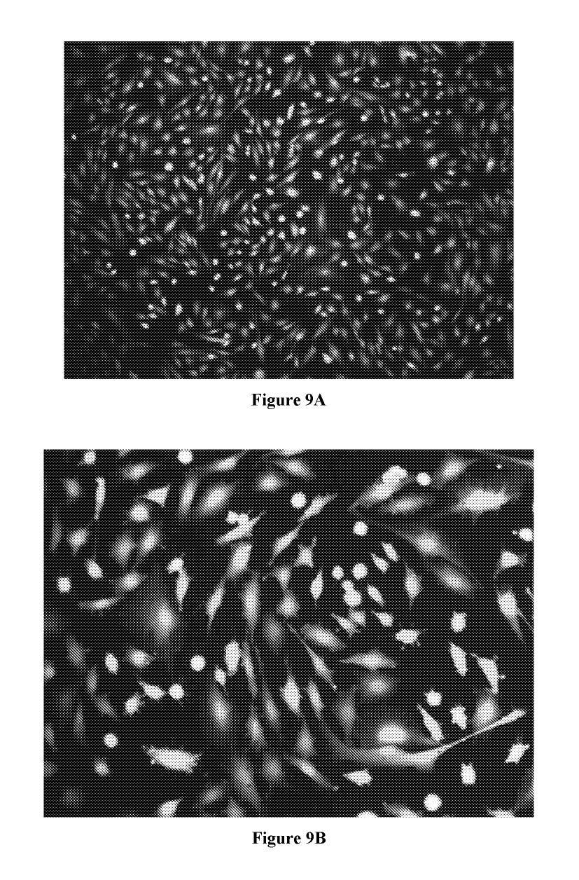

Next, the viability of the electroporated cells was verified through visual detection of cultured cell growth. More specifically, as illustrated in FIGS. 8A-B, the cultured cells demonstrated grown at both 24 hours (FIG. 8A) and 48 hours (FIG. 8B) post electroporation. Turning to FIGS. 9A-B, it was also demonstrated that the protein remained in the cells for at least 48 hours following electroporation.

### Example 5: mRNA Coding for Proteins Targeting Cancer Cells

To demonstrate low-cost CAR-modified T-cell production without on-target/off-tumor toxicity, T-cells are manufactured to express ErbB2 (common target for cancer). Expression of ErbB2 has been used in the past but not as the result of the device or methods of the present disclosure. Green fluorescent protein (GFP) mRNA is used as the positive control and Anti-erbB2-scFv-CD28-OX40-CD3ζ mRNA targeting the production of ErbB2 immunoreceptors as the experiment. Each experiment involving T-cells is performed for CD4⁺ and CD8⁺ T-cells separately. Creative Biolabs (New York, N.Y.), a leading company for the production of CAR for adoptive immunotherapy, generates retroviral CAR vector for ErbB2 mRNA. First, the quantitative mRNA injection into T-cells through electroporation is studied, followed by the correlation between electroporation time and the average expression of immunoprotein in single T-cells (through fluorescence). Finally, the technology is scaled up to produce 10⁷ T-cells cost-effectively.

### Example 6: Determine a Correlation Between Electroporation Time and Immunoreceptor Expression Level in T-Cells. in Chamber 2

Although electroporation has been utilized to transfect T-cells with mRNA molecules, no study has focused on quantitatively transfecting T-cells with mRNA molecules. The objective of this example is to utilize electroporation for quantitative transfection of T-cells with mRNA molecules. As mentioned, there must be a dependence relationship between electroporation time and immunoreceptor expression. To demonstrate the feasibility, a relationship between electroporation time and expression level of immunoreceptor in single T-cells was determined. More specifically, T-cell patterning between IDEs was utilized and electroporation was performed on T-cells for a desired time. This provided a relationship between electroporation time and number of immunoreceptors on T-cells. The distribution of immunoreceptors on T-cells was also studied, using about 10⁶ T-cells per experiment.

Without wishing to be bound by theory, it is believed that when other conditions do not vary, the number of mRNA molecules transported into T-cells will depend on electroporation time. Since the expression levels of immunoreceptor molecules are proportional to the number of mRNA molecules, immunoreceptor expression level in T-cells can be controlled thorough the electroporation time.

Dielectrophoresis (DEP):

The DEP is the movement of T-cells relative to the buffer, resulting from polarization forces produced by non-uniform AC (alternating current) electrical fields. Mathematically, the time-average DEP force acting on a T-cell in a non-uniform AC electric field can be represented by

F_{DEP}=½α∇(E²) (1)

where α is the polarizability of the T-cell, ∇ is the vector operator, and E is the rms electric field, also known as the equivalent DC value.

α=4πεₘr³ Re{f_{CM}(ω)}, (2)

where r is the radius of the T-cell, εₘ is the suspending medium permittivity, ω is the frequency of the applied electric field, and Re{f_{CM}(ω)} is the real part of the Clausius-Mossotti factor is defined as

f_{CM}(ω)=(εₚ*−εₘ*)/(εₚ*+2εₘ*) (3)

where εₚ* is the complex permittivity of the T-cell and εₘ* is the complex permittivity of the suspending medium. The complex permittivity is given by

$${ɛ}^{*} = ɛ - j ( \frac{σ}{ω} )$$

with σ the real conductivity, ε the real permittivity and ω is the frequency. The real part of the Clausius-Mossotti factor is theoretically bounded between −½ and 1, which determines the direction and the relative strength of the DEP force. If the magnitude of Re{f_{CM}(ω)} is negative, then the T-cells move towards the lowest field strength region (negative DEP) and if it is positive T-cells are repelled from the lowest field strength region and move to regions of highest field strength (positive DEP).

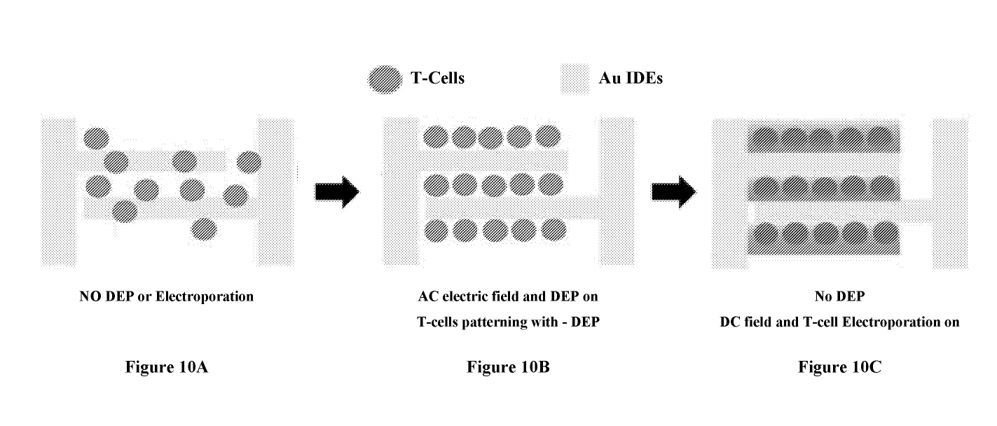

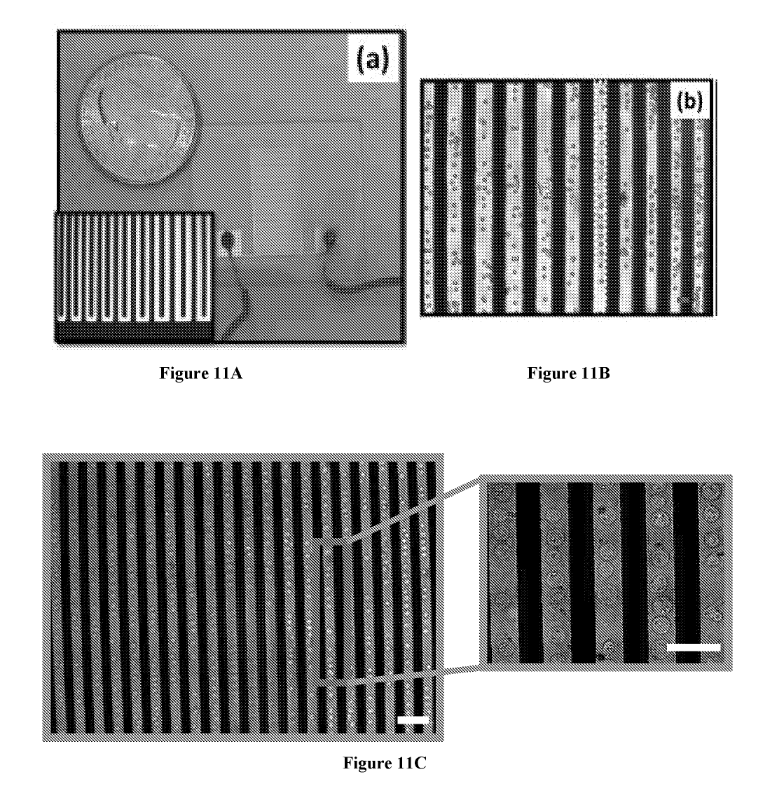

FIG. 2 illustrates the basic steps of the production of CAR T-cells. FIGS. 10A-B show the purification of the expanded T-cells. The T-cells are trapped on IDE (micro-interdigitated electrodes) using DEP and wash away other materials such as beads and medium. Finally FIG. 10C shows the electroporation of patterned T-cells.

The magnitude of induced electric potential (electric field) in T-cell membranes determines the size of the pore that is used to transport exogenous mRNA molecules through electroporation. Therefore, to produce a homogenous mixture of T-cells transfected with mRNA molecules, it is required to have a uniform induced electric field in all T-cell membranes in the cell mixture. To experimentally achieve this, T-cells were first patterned as single files between parallel micro-electrodes so that every single T-cell will be subjected to the same external electric field. This was achieved utilizing micro-interdigitated electrodes (IDE) and T-cell patterning through the use of DEP. FIG. 11A illustrates the IDEs designed and fabricated for cell patterning/electroporation experiments. Standard lithography based microfabrication was used to fabricate IDE in Au on glass substrates. The space between electrodes was set to 30 μm to accommodate a single file of T-cells between electrodes.

Since the T-cell patterning is needed prior to electroporation, DEP based cell patterning on IDEs was demonstrated using commercially available polystyrene beads and C2C12 cells (mouse myoblast cells). The polystyrene beads were used in initial experiments as they are convenient to use and to find basic experimental conditions. The C2C12 and T-cells have identical dielectric properties and therefore C2C12 provide a good model to study T-cells. The process for DEP-based cell patterning is simple: first, cultured cells were suspended in standard 1×PBS buffer and about 500 μL of cells and buffer was pipetted onto the IDE electrode area. An AC electric field (10 Vpp and 120 kHz) was applied and patterned the cells using negative DEP (FIGS. 11B-C). The frequency and magnitude of the electric field was then varied to achieve the successful T-cell patterning. Under these electric field conditions, more than 99% of the T-cells on the electrodes were patterned. Finally, the electric field (DEP) was turned off and it was observed that patterned T-cells neither move nor are disturbed.

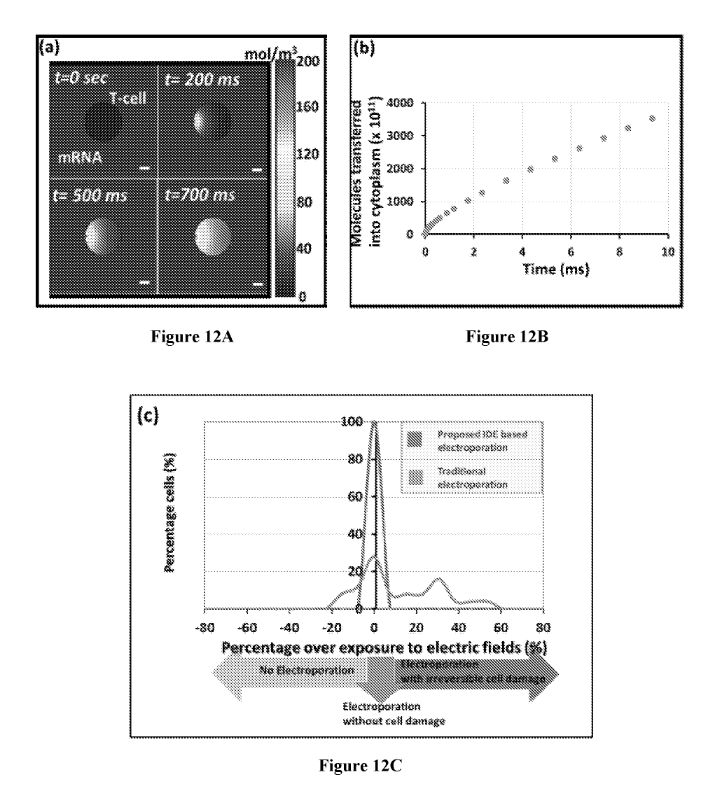

It has been reported that about 1.25×10⁵ V/m induced electric field in the T-cell membrane is required to electroporate T-cells. To study how T-cell patterning helps uniformly induce electric fields in each T-cell in a pattern, the induced electric fields on the membranes of every single T-cell that are in a pattern was calculated utilizing COMSOL software. In this calculation, a sample of 25 cells was used and a short DC electric pulse (1V) between IDEs was applied. FIG. 12C—red curve illustrates the induced electric fields on the membranes of patterned T-cells and the blue curve indicates the calculated induced electric fields of T-cells that are in traditional, commercially available electroporation equipment. By simple comparison, it is concluded that the proposed T-cell patterning based electroporation induces uniform electric fields in all T-cells and therefore all the T-cells will be transfected with equal amounts of mRNA molecules. In comparison, with current electroporation devices used in T-cell engineering (FIG. 12C—blue line), only about 20% of T-cells are electroporated with proper electric fields and 50% of T-cells are exposed to extremely high electric fields causing irreversible cell damage. Approximately 10% of cells are not electroporated at all. Out of 50% of T-cells that exposing to high electric fields, about 5-10% of T-cells will be viable. Therefore, T-cell viability number will be about 40% (20%+10%+5-10%). This agrees with published work by others.

A correlation between electroporation time and immunoreceptor expression level of single T-cells for GFP and ErbB2 mRNA molecules was determined by conducting the following experimental and theoretical studies:

Finding the Frequency and the Magnitude of the Electric Field Needed for Repeatable T-Cell Patterning Between IDEs.

The IDE array used in preliminary studies was used as the starting point of T-cell patterning. The instant inventors have demonstrated the successful cell patterning on IDEs using C2C12 cells (FIGS. 11A-C). Using these results as the basis, T-cells were patterned on IDEs. It has been reported that T-cells experience a large DEP around 100 kHz. Since the DEP force is utilized to pattern T-cells on IDEs, T-cell patterning began at 100 kHz and the patterning for 50-250 kHz in 50 kHz steps was studied. For each frequency, the number of T-cells in patterns as single files was studied between individual IDEs. The frequency that generates T-cell patterns with the highest number of T-cells was chosen. Additionally, the effect of T-cell concentration, magnitude of the electric field, physical gap between electrodes (30 μm), and conductivity of the T-cell medium on the T-cell patterning was studied. Each of these experimental conditions is studied separately to find the optimum experimental conditions (frequency, magnitude of the electric field, gap between individual IDE pairs) needed for a repeatable T-cells pattern with single file of T-cells between each IDE pair. Although it has been reported that exposure to low frequency electric fields/electric field gradients do not alter the cellular chemistry or cell viability, the instant inventors also studied the cell viability with the optimized experimental condition. The T-cell viability was studied using a commercially available assay kit (Cell Titer 96 ®, Promega, Madison, Wis.).

Determine the Magnitude of the DC Electric Field Needed to Produce a Homogenous Mixture Immunoreceptor Expressing T-Cells:

According to the Nernst-Planck model, the mRNA transport into T-cells depends on the magnitude of the electric pulse (pulse height) that are applied for electroporation. In this example, the electroporation time is kept as a constant (pulse width˜5 ms, typical number in T-cell) and the mRNA transport is studied with the pulse height (magnitude of the DC field). Commercially available mRNA electroporation equipment (Genepulser Xcell, Munich, Germany) use 2×10⁵ V/m (500V across electrodes separated by 4 mm). Therefore, the instant inventors use 0.5, 1, 1.5, 2, 2.5, 3 and 4×10⁵ V/m to transfect T-cells with GFP mRNA molecules. Briefly, the T-cells are first patterned on IDEs using the experimental condition obtained above. The DEP is then turned off and DC pulses (indicated above) are applied for 5 ms. After each electroporation, a T-cell sample is collected and suspended in RPMI 1640 and incubated for 4-6 hours. The GFP expression is then measured through standard fluorescence activated cell sorting (FACS). The cell count vs. fluorescence intensity plotted and the distribution of the GFP in each T-cells sample produced from each electric field is analyzed. For each pulse height, the fluorescence vs. cell count is studied. Finally, the height of the DC pulse that produces the narrowest fluorescence width is determined. The condition that has the narrowest fluorescence width produces the homogenous mixture of T-cells with GFP expressions.

Determining a Correlation Between Electroporation Time and Expression Levels of Immunoreceptors in T-Cells.

When all other electroporation conditions are optimized, according to the Nernst-Planck model, for a given initial concentration of mRNA molecules in the electroporation buffer, the electroporation time determines the number of mRNA transported into T-cells (FIG. 12B). The experimental conditions above may be used to find a correlation between electroporation time and expression of immunoreceptors in T-cells. First, about 10⁶ T-cells are patterned in the IDE array using the conditions obtained above. With 200 μg/mL of GFP mRNA molecules in the buffer, the DEP force is turned off and DC electric pulse obtained above is applied for ˜5 ms for the IDEs array, and the T-cells are transfected with mRNA molecules. After the electroporation, T-cells are collected from the IDE array and suspended in RPMI 1640 media for 4-6 hours, followed by measurement of the mean fluorescence intensity (MFI) per T-cell. As the next step, the electroporation time is changed from 100 μs to 10 ms in 500 μs intervals and the mean fluorescence intensity (MFI) per T-cell is determined for each condition. After changing electroporation time, the MFI per T-cell is converted to number of GFP protein molecules using commercially available reagent kit (QIFIKIT, Dako, Carpinteria, Calif.). The electroporation time and expression of GFP molecules is then plotted to find a correlation. Finally the experiments are repeated using ErbB2 mRNA.

Accomplishment of the tasks in this Example generates knowledge on how T-cells interact with low frequency electric fields and produce electroporation. This knowledge is utilized to quantitatively inject mRNA molecules into T-cells to produce the required level of immunoreceptors, thereby accomplishing the objective of this aim.

When there is a large variation in expression levels of immunoreceptor from experiment to experiment, multiple regression analysis may be utilized to determine the correlation between electroporation time and the immunoreceptor expression level. Multiple regression analysis has been successfully utilized in similar studies (i.e. to analyze mRNA-protein relationships) by others and has been shown to be useful. Furthermore, if there is not T-cell patterning with DEP, electrophoresis-based T-cell pattering may be used. Starting with small electric fields (100 mV in 30 μm spacing IDEs), the optimum electric field needed for successful T-cell patterning is determined.

### Example 7: Quantify the Dynamics of Tumor Cell Cytotoxicity and the Expression of Immunoreceptors in T-Cells. in Chamber 2

Expression of immunoreceptors in T-cells is needed to lyse tumor-cells. Over-expression will increase the probability of the interaction between T-cells with healthy tissue cells, resulting in on-target/off-tumor toxicity. Therefore, it is required to find the minimum immunoreceptor expression level needs to be presented in T-cells to sufficiently lyse all target tumor cells. This will also be the safest level of immunoreceptor expression to minimize the on-target/off-tumor toxicity. This ability also leads into the study of the immunoreceptor expression dependent toxicity. This minimum level will depend on factors such as tumor type, stage, and the person. Once the minimum level of the immunoreceptors is determined, the production of T-cells with desired level of expression is performed.

This Example demonstrate the methodology used to find the minimum expression level of the immunoreceptor molecules in T-cells. These minimum expression levels may be used to develop a chart of minimum immunoreceptor molecules needed based on the tumor type, stage, age of the patient, etc., so that users can choose the appropriate values to produce T-cells. In addition, one can use the instant device to find the appropriate minimum expression level.

To successfully quantify the dynamics of tumor cell cytotoxicity and the expression of immunoreceptors in T-cells, when the electroporation has been completed, mRNA molecules that are in T-cells must freely scatter in the cytoplasm. The scattering of mRNA molecules is needed to avoid the unnecessary cross-hybridization between mRNA molecules. The cross hybridization may prevent the translation of mRNA molecules into immunoreceptors. Through calculations using the Nernst-Planck equation, the instant inventors have demonstrated that the mRNA molecules will indeed scatter in the cytoplasm (FIG. 12A). Therefore, it can be concluded that mRNA molecules that are transported into T-cells through electroporation will produce immunoreceptors on T-cell surfaces. Furthermore, the instant inventors have demonstrated that exposing mRNA molecules to low frequency electric fields (<150 kHz) and high electric field gradients (˜10¹⁵ V/m²) does not damage or alter the biological functionality.

Accordingly, the inventors next determined how to control the number of mRNA molecules that are transported into T-cells. Since the mRNA molecules are large (>4 kDa), it has been theoretically demonstrated that mRNA molecules will not efficiently transport into T-cells through simple diffusion. It has been experimentally observed that the transport mechanism is through electric field mediated diffusion (electro-diffusion). To fully understand this process, the Nernst-Planck equation was used to model the mRNA transport into a T-cell through electro-diffusion. It describes the motion of charged particles in fluid. Calculations were performed using COMSOL software and FIG. 12A illustrates the snap shots of the mRNA transport through electro-diffusion of an electroporated T-cell.

To understand the controlled mRNA transport into T-cells, the mRNA molecule transport into a single T-cell was studied with time. When time T=0 secs, the electroporation was started and counting the number of mRNA molecules that are being transported into the T-cell (with time) began (FIG. 12B). In this calculation, an initial mRNA molecule concentration of 200 μg/mL (typical concentration use to electroporate T-cells) was assumed. This mRNA concentration was used in current electroporation based T-cell production techniques. Through this calculation, it was demonstrated that there is a proportional relationship between the number of mRNA molecules that were transported into T-cells and the electroporation time. Therefore, by changing the initial mRNA concentration in the buffer and electroporation time, the number of mRNA molecules transported into T-cells can be precisely controlled. These results strongly support the belief that cytotoxicity of T-cells toward tumor cells will depend on the number of immunoreceptors expressed in T-cells.

Methods

Determining the Minimum Expression Level of Immunoreceptors to Effectively Lyse Tumor Cells.

The ErbB2 mRNA molecules are injected into T-cells (CD8⁺, about 10⁶ cells per run) by following the experimental conditions determined in Example 3. Briefly, 200 μg/mL ErbB2 mRNA molecules are used in the electroporation buffer and eight experiments are performed by changing the electroporation time (electroporation time=100, 250, 500, 750, 1000, 1500, 2000 and 3000 μs). Each experiment is repeated three times and the statistical significance is calculated. After electroporation, T-cells are collected and suspended in the RPMI 1640 separately and incubated for 4-6 hours to produce immunoreceptors. A cytotoxicity assay is then performed to analyze the transfected CD8⁺ T-cells' ability to lyse target tumor cells. Commercially available human ovary cancer cells (SKOV3) are used as the target cells. It has been demonstrated that ErbB2 modified T-cells lyse SKOV3 cells. The cell lysis is accomplished using a commercially available kit (CellTrase CFSE kit, Thermo Fisher scientific). Briefly, T-cells transfected with mRNA molecules are activated using Muromonab-CD3 (OKT3, 100 ng/ml) and Interleukin-2 (IL-2, 400 U/ml) for two days. Tumor cells (ratio 1:2=SKOV3: T-cells (CD8⁺)) are then added, incubate for 48 hours, and the proliferation assay is performed. The variation of the tumor cells' viability is calculated and compared with each electroporation time and the minimum electroporation time needed for lysing all tumor cells is determined. The electroporation time is converted to find the number of immunoreceptors needed in T-cells. This immunoreceptor level is called the “lytic threshold.” Negative control experiments are performed using MDAMB 468 (ATCC, Manassas, Va.) where no cell lysis is expected.

Determining the Minimum Expression Level of Immunoreceptors Needed in T-Cells to Secrete Cytokines.

To determine minimum expression level needed to secrete cytokines, T-cell samples of CD4⁺ and CD8⁺ cells (10⁶ cells) separately with ErbB2 mRNA molecules (200 μg/mL) were electroporated. For each T-cell type, the electroporation time was changed from 100, 250, 500, 750, 1000, 1500, 2000 and 3000 μs and eight samples were produced. Each T-cell type is mixed with SKOV3 (1:1 ratio 500000:500000 cells) and the mixture is incubated for 16-20 hours to study the cytokine release. It has been reported that 16-20 hours incubation will be sufficient to release cytokines. After incubation, the concentrations of common cytokines (interleukin 2 (IL-2), tumor necrosis factor (TNFα), and interferon-γ (IFNγ)) in ng/mL in each cell sample is determined using a commercially available cytokine assays kit and following the manufacturer's protocols (BD Biosciences). In parallel, T-cell samples (CD4⁺ and CD8⁺ cells) that did not undergo electroporation to transfect with ErbB2 are assayed. These experiments may be used as the negative control experiments.

The concentrations of IL-2, TNFα and IFNγ are then compared in each T-cell sample produced using various electroporation times with the negative control. The analysis of variance (ANOVA) is used to compare the cytokine concentrations and determine the minimum electroporation time needed to secrete the cytokine. The electroporation time is converted to number immunoreceptors needed in T-cells (CD4⁺ or CD8⁺) to release cytokine molecules. This immunoreceptor level is called the “activating threshold.”

In the method above, CD4⁺ and CD8⁺ cells are used separately, and the best option is selected and compared with the results for effective tumor cell lysing above.

Determining the Minimum Expression Level of Immunoreceptors Needed to Effectively Lyse the Target Cells and Release Cytokines while Minimizing the on Target/Off Tumor Toxicity.

It has been reported that the lytic and activating thresholds have two different thresholds. Published studies involving lymphoblastic leukemia and chronic lymphocytic leukemia have reported that the activating threshold is a few thousand molecules (per T-cell) higher than the lytic threshold. Furthermore, it has been theorized that these threshold values are dependent on the tumor type, stage and the patient.

Here, these two threshold values are compared. These experiments were performed using the heterogeneous mixtures of immunoreceptor expressing T-cells and therefore true comparison between the two conditions is difficult and results may not be accurate. If the lytic threshold is greater than the activating threshold, the lytic threshold is used and the T-cells are electroporated to produce the immunoreceptor level that is equal to the lytic threshold. Alternatively, if the activating threshold is greater, T-cells are electroporated to produce the immunoreceptor level equal to the activating threshold. Since this is the minimum level of the immunoreceptors in T-cells to effectively eliminate the tumor cells, this is the safest level of immunoreceptor expression needed for proper efficacy. As the next step, the lytic and activating thresholds of two other ErbB2 positive tumor cells (breast cancer cells (MDA-MB-361) and lung cancer cells (A549)) are examined and studied. These cell-lines are purchased from ATCC (Manassas, Va.) and cultured in the lab. The procedures developed are used to experimentally find the lytic and activating thresholds. This data is analyzed and conclusions may be made based on the tumor type.

This Example yields knowledge on the fundamental relationship between immunoreceptor expression and lytic/activating thresholds in a wide range of tumor cells. This knowledge is used to achieve the main objective of this Example.

### REFERENCES

[1] Brenner, Malcolm K., and Helen E. Heslop. “Adoptive T cell therapy of cancer.” Current opinion in immunology 22.2 (2010): 251-257.
[2] Kalos, Michael, and Carl H. June. “Adoptive T cell transfer for cancer immunotherapy in the era of synthetic biology.” Immunity 39.1 (2013): 49-60.
[3] Kalos, Michael, et al. “T cells with chimeric antigen receptors have potent antitumor effects and can establish memory in patients with advanced leukemia.” Science translational medicine 3.95 (2011): 95ra73-95ra73.
[4] O'connell, Joe, et al. “The Fas counterattack: Fas-mediated T cell killing by colon cancer cells expressing Fas ligand.” The Journal of experimental medicine 184.3 (1996): 1075-1082.
[5] Wang, Xiuyan, and Isabelle Rivière. “Clinical manufacturing of CAR T cells: foundation of a promising therapy.” Molecular Therapy Oncolytics 3 (2016): 16015.
[6] the-scientist.com/?articles.view/articleNo/42462/title/The-CAR-T-Cell-Race/
[7] Watanabe, Keisuke, et al. “Target Antigen Density Governs the Efficacy of Anti-CD20-CD28-CD3 ζ Chimeric Antigen Receptor-Modified Effector CD8+ T Cells.” The Journal of Immunology 194.3 (2015): 911-920.
[8] Park, Tristen S., Steven A. Rosenberg, and Richard A. Morgan. “Treating cancer with genetically engineered T cells.” Trends in biotechnology 29.11 (2011): 550-557.
[9] Gascoyne, Peter RC, and Jody Vykoukal. “Particle separation by dielectrophoresis.” Electrophoresis 23.13 (2002): 1973.
[10] Markx, Gerard H., Mark S. Talary, and Ronald Pethig. “Separation of viable and non-viable yeast using dielectrophoresis.” Journal of Biotechnology 32.1 (1994): 29-37.
[11] Birkholz, K., Hombach, A., Krug, C., Reuter, S., Kershaw, M., Kämpgen, E., Schuler, G., Abken, H., Schaft, N. and Dörrie, J., Transfer of mRNA encoding recombinant immunoreceptors reprograms CD4&plus; and CD8&plus; T cells for use in the adoptive immunotherapy of cancer. Gene therapy, 16(5), pp. 596-604, 2009.
[12] Barrett, David M., et al. “Treatment of advanced leukemia in mice with mRNA engineered T cells.” Human gene therapy 22.12 (2011): 1575-1586.
[13] Cheadle, Eleanor J., et al. “Chimeric antigen receptors for T-cell based therapy.” Antibody Engineering: Methods and Protocols, Second Edition (2012): 645-666.
[14] Barrett, D. M., Zhao, Y., Liu, X., Jiang, S., Carpenito, C., Kalos, M., Carroll, R. G., June, C. H. and Grupp, S. A., Treatment of advanced leukemia in mice with mRNA engineered T cells. Human gene therapy, 22(12), pp. 1575-1586, 2011.
[15] Jordan, C. A., Neumann, E. and Sowers, A. E. eds., Electroporation and electrofusion in cell biology. Springer Science & Business Media., 2013.
[16] Isambert, H., Understanding the electroporation of cells and artificial bilayer membranes. Physical review letters, 80(15), p. 3404, 1998.
[17] Bonifant, C. L., Jackson, H. J., Brentjens, R. J. and Curran, K. J., Toxicity and management in CAR T-cell therapy. Molecular Therapy Oncolytics, 3, 2016.
[18] Davila, M. L., Riviere, I., Wang, X., Bartido, S., Park, J., Curran, K., Chung, S. S., Stefanski, J., Borquez-Ojeda, O., Olszewska, M. and Qu, J., Efficacy and toxicity management of 19-28z CAR T cell therapy in B cell acute lymphoblastic leukemia. Science translational medicine, 6(224), pp. 224ra25-224ra25, 2014.
[19] Friedrich, G. and Soriano, P., Promoter traps in embryonic stem cells: a genetic screen to identify and mutate developmental genes in mice. Genes & development, 5(9), pp. 1513-1523, 1991.
[20] Valero, A., Post, J. N., Van Nieuwkasteele, J. W., Ter Braak, P. M., Kruijer, W. and Van Den Berg, A., Gene transfer and protein dynamics in stem cells using single cell electroporation in a microfluidic device. Lab on a Chip, 8(1), pp. 62-67, 2008.
[21] Huang, Ying, et al. “Dielectrophoretic cell separation and gene expression profiling on microelectronic chip arrays.” Analytical chemistry 74.14 (2002): 3362-3371.
[22] Li, Y., Dalton, C., Crabtree, H. J., Nilsson, G., & Kaler, K. V. (2007). Continuous dielectrophoretic cell separation microfluidic device. Lab on a Chip, 7(2), 239-248.
[23] Sugar, I. P., & Neumann, E. (1984). Stochastic model for electric field-induced membrane pores electroporation. Biophysical chemistry, 19(3), 211-225.
[24] Sukharev, S. I., Klenchin, V. A., Serov, S. M., Chernomordik, L. V., & YuA, C. (1992). Electroporation and electrophoretic DNA transfer into cells. The effect of DNA interaction with electropores. Biophysical journal, 63(5), 1320.
[25] Mittal, Nikhil, Adam Rosenthal, and Joel Voldman. “nDEP microwells for single-cell patterning in physiological media.” Lab on a Chip 7.9 (2007): 1146-1153.
[26] Lin, R. Z., Ho, C. T., Liu, C. H., & Chang, H. Y. (2006). Dielectrophoresis based-cell patterning for tissue engineering. Biotechnology journal, 1(9), 949-957.
[27] Kim, S. K., Hesketh, P. J., Li, C., Thomas, J. H., Halsall, H. B., & Heineman, W. R. (2004). Fabrication of comb interdigitated electrodes array (IDA) for a microbead-based electrochemical assay system. Biosensors and Bioelectronics, 20(4), 887-894.
[28] Muratore, M., Mitchell, S., & Waterfall, M. (2013). Plasma membrane characterization, by scanning electron microscopy, of multipotent myoblasts-derived populations sorted using dielectrophoresis. Biochemical and biophysical research communications, 438(4), 666-672.
[29] DeBruin, K. A., & Krassowska, W. (1999). Modeling electroporation in a single cell. II. Effects of ionic concentrations. Biophysical journal, 77(3), 1225-1233.
[30] Krassowska, W., & Filev, P. D. (2007). Modeling electroporation in a single cell. Biophysical journal, 92(2), 404-417.
[31] Vasilkoski, Z., Esser, A. T., Gowrishankar, T. R., & Weaver, J. C. (2006). Membrane electroporation: the absolute rate equation and nanosecond time scale pore creation. Physical review E, 74(2), 021904.
[32] Nawarathna, D., Unal, K., & Wickramasinghe, H. K. (2008). Localized electroporation and molecular delivery into single living cells by atomic force microscopy. Applied Physics Letters, 93(15), 153111.
[33] Nawarathna, D., Turan, T., & Wickramasinghe, H. K. (2009). Selective probing of mRNA expression levels within a living cell. Applied physics letters, 95(8), 083117.
[34] Dieckmann, D., Plottner, H., Berchtold, S., Berger, T. and Schuler, G., Ex vivo isolation and characterization of CD4+ CD25+ T cells with regulatory properties from human blood. The Journal of experimental medicine, 193(11), pp. 1303-1310, 2001.
[35] Dorsam, G., Graeler, M. H., Seroogy, C., Kong, Y., Voice, J. K. and Goetzl, E. J., Transduction of multiple effects of sphingosine 1-phosphate (SIP) on T cell functions by the S1P1 G protein-coupled receptor. The Journal of lmmunology, 171(7), pp. 3500-3507, 2003.
[36] Germishuizen, W. A., Wälti, C., Wirtz, R., Johnston, M. B., Pepper, M., Davies, A. G. and Middelberg, A. P., 2003. Selective dielectrophoretic manipulation of surface-immobilized DNA molecules. Nanotechnology, 14(8), p. 896, 2003.
[37] Boote, J. J. and Evans, S. D., Dielectrophoretic manipulation and electrical characterization of gold nanowires. Nanotechnology, 16(9), p. 1500, 2005.
[38] Lapizco-Encinas, B. H. and Rito-Palomares, M., Dielectrophoresis for the manipulation of nanobioparticles. Electrophoresis, 28(24), pp. 4521-4538, 2007.
[39] Hölzel, R., Calander, N., Chiragwandi, Z., Willander, M. and Bier, F. F., Trapping single molecules by dielectrophoresis. Physical review letters, 95(12), p. 128102, 2005.
[40] Riet, T., Holzinger, A., Dörrie, J., Schaft, N., Schuler, G., & Abken, H. (2013). Nonviral RNA transfection to transiently modify T cells with chimeric antigen receptors for adoptive therapy. Synthetic Messenger RNA and Cell Metabolism Modulation: Methods and Protocols, 187-201.
[41] Winkler, C., Steingrube, D. S., Altermann, W., Schlaf, G., Max, D., Kewitz, S., . . . & Staege, M. S. (2012). Hodgkin's lymphoma RNA-transfected dendritic cells induce cancer/testis antigen-specific immune responses. Cancer Immunology, Immunotherapy, 61(10), 1769-1779.
[42] Orfao, A., & Ruiz-Argüelles, A. (1996). General concepts about cell sorting techniques. Clinical biochemistry, 29(1), 5-9.
[43] Gygi, S. P., Rist, B., Gerber, S. A., Turecek, F., Gelb, M. H. and Aebersold, R., Quantitative analysis of complex protein mixtures using isotope-coded affinity tags. Nature biotechnology, 17(10), pp. 994-999, 1999.
[44] Gygi, S. P., Rochon, Y., Franza, B. R. and Aebersold, R., Correlation between protein and mRNA abundance in yeast. Molecular and cellular biology, 19(3), pp. 1720-1730, 1999.
[45] Nicholson, J. K., Connelly, J., Lindon, J. C., & Holmes, E. (2002). Metabonomics: a platform for studying drug toxicity and gene function. Nature reviews Drug discovery, 1(2), 153-161.
[46] Fu, A. Y., Chou, H. P., Spence, C., Arnold, F. H., & Quake, S. R. (2002). An integrated microfabricated cell sorter. Analytical Chemistry, 74(11), 2451-2457.
[47] Hombach, A., Wieczarkowiecz, A., Marquardt, T., Heuser, C., Usai, L., Pohl, C., . . . & Abken, H. (2001). Tumor-specific T cell activation by recombinant immunoreceptors: CD3ζ signaling and CD28 costimulation are simultaneously required for efficient IL-2 secretion and can be integrated into one combined CD28/CD3ζ signaling receptor molecule. The Journal of Immunology, 167(11), 6123-6131.
[48] Ohigashi, Y., Sho, M., Yamada, Y., Tsurui, Y., Hamada, K., Ikeda, N.,& Tsushima, F. (2005). Clinical significance of programmed death-1 ligand-1 and programmed death-1 ligand-2 expression in human esophageal cancer. Clinical Cancer Research, 11(8), 2947-2953.
[49] Barrett, D. M., Zhao, Y., Liu, X., Jiang, S., Carpenito, C., Kalos, M., . . . & Grupp, S. A. (2011). Treatment of advanced leukemia in mice with mRNA engineered T cells. Human gene therapy, 22(12), 1575-1586.
[50] Fraietta, J. A., Schwab, R. D., & Maus, M. V. (2016, April). Improving therapy of chronic lymphocytic leukemia with chimeric antigen receptor T cells. In Seminars in oncology (Vol. 43, No. 2, pp. 291-299). WB Saunders.
[51] Kim, M. G., Kim, D., Suh, S. K., Park, Z., Choi, M. J., & Oh, Y. K. (2016). Current status and regulatory perspective of chimeric antigen receptor-modified T cell therapeutics. Archives of pharmacal research, 39(4), 437-452.
[52] Moulder, S. L., Yakes, F. M., Muthuswamy, S. K., Bianco, R., Simpson, J. F., & Arteaga, C. L. (2001). Epidermal growth factor receptor (HER1) tyrosine kinase inhibitor ZD1839 (Iressa) inhibits HER2/neu (erbB2)-overexpressing breast cancer cells in vitro and in vivo. Cancer research, 61(24), 8887-8895.
[53] Pantel, K., Brakenhoff, R. H., & Brandt, B. (2008). Detection, clinical relevance and specific biological properties of disseminating tumour cells. Nature Reviews Cancer, 8(5), 329-340.
[54] Qin, Dong, Younan Xia, and George M. Whitesides. “Soft lithography for micro- and nanoscale patterning.” Nature protocols 5, no. 3 (2010): 491-502.
[55] Quake, S. R., & Scherer, A. (2000). From micro- to nanofabrication with soft materials. Science, 290(5496), 1536-1540.
[56] Sia, Samuel K., and George M. Whitesides. “Microfluidic devices fabricated in poly (dimethylsiloxane) for biological studies.” Electrophoresis 24, no. 21 (2003): 3563-3576.
[57] Stroock, A. D., & Whitesides, G. M. (2002). Components for integrated poly (dimethylsiloxane) microfluidic systems. Electrophoresis, 23(20), 3461-3473.
[58] J. Schrama, Thesis, University of Leiden, Leiden, Netherlands, 1957.
[59] Krug, C., Wiesinger, M., Abken, H., Schuler-Thurner, B., Schuler, G., Dörrie, J., & Schaft, N. (2014). A GMP-compliant protocol to expand and transfect cancer patient T cells with mRNA encoding a tumor-specific chimeric antigen receptor. Cancer Immunology, Immunotherapy, 63(10), 999-1008.
[60] Dörrie, J., Krug, C., Hofmann, C., Müller, I., Wellner, V., Knippertz, I., . . . & Fritsch, G. (2014). Human Adenovirus-Specific γ/δ and CD8+ T Cells Generated by T-Cell Receptor Transfection to Treat Adenovirus Infection after Allogeneic Stem Cell Transplantation. PloS one, 9(10), e109944.
[61] Höfflin, S., Prommersberger, S., Uslu, U., Schuler, G., Schmidt, C. W., Lennerz, V., . . . & Schaft, N. (2015). Generation of CD8+ T cells expressing two additional T-cell receptors (TETARs) for personalised melanoma therapy. Cancer biology & therapy, 16(9), 1323-1331.
[62] bls.gov/ooh/architecture-and-engineering/biomedical-engineers.htm.
[63] Jeffery Mervis, (2016), Genuine research keeps students in science, Science, 352(6291), 1266.
[64] Brainard, S. G., & Carlin, L. (1997, November). A longitudinal study of undergraduate women in engineering and science. In Frontiers in Education Conference, 1997. 27th Annual Conference. Teaching and Learning in an Era of Change. Proceedings. (Vol. 1, pp. 134-143). IEEE.
[65] Eccles, J. S. Where Are All the Women? Gender Differences in Participation in Physical Science and Engineering. American Psychological Association, (2007).
[66] McIlwee, J. S., & Robinson, J. G. Women in engineering: Gender, power, and workplace culture. SUNY Press, (1992).
[67] Heaverlo, C., STEM Development: A Study of 6th-12th Grade Girls' Interest and Confidence in Mathematics and Science, PhD Dissertation, Iowa State University, 2011.
[68] Rabenberg, T. A., Middle School Girls' STEM Education: Using Teacher Influences, Parent Encouragement, Peer Influences, And Self Efficacy To Predict Confidence And Interest In Math And Science (Doctoral dissertation, Drake University), 2013.
[69] Riet, T., Holzinger, A., Dörrie, J., Schaft, N., Schuler, G., & Abken, H. (2013). Nonviral RNA transfection to transiently modify T cells with chimeric antigen receptors for adoptive therapy. Synthetic Messenger RNA and Cell Metabolism Modulation: Methods and Protocols, 187-201.
[70] Anurathapan, U., Leen, A. M., Brenner, M. K., & Vera, J. F. (2014). Engineered T cells for cancer treatment. Cytotherapy, 16(6), 713-733.

1. A device to produce modified T-cells comprising a first chamber for proliferating a population of T-cells and a second chamber for modifying the T-cells to express a desired T-cell receptor antigen.

2. A device according to claim 1 wherein each of chamber 1 and chamber 2 comprise a top surface, a bottom surface and four sides.

3. A device according to claim 2 wherein two sides are essentially parallel to each other and are the ends and another two sides are essentially parallel and are the sides.

4. A device according to claim 2 wherein the top and bottom surface are parallel to each other and are at a distance of about 100 μm.

5. A device according to claim 3 wherein one or both ends have openings.

6. A device according to claim 2 wherein the top and the side surfaces are made of PDMS and the bottom surface is made of glass.

7. A device according to claim 1 wherein chamber one consists of a series of T-traps.

8. A device according to claim 1 wherein chamber 2 comprises one or more channels bordered on each side by a metal, wherein the metal on one side is connected to one electrical pole and the other side is connected to a second electrical pole to allow an electrical potential to flow between the two poles.

9. A device according to claim 7 wherein the metal is selected from the group consisting of gold (Au), silver (Ag), platinum (Pt), copper (Cu), iron (Fe), aluminum (Al), Tin (Sn), Nickel (Ni), Carbon (C), doped silicon (Si) or an alloy of metals.

10. A device according to claim 8 wherein the metal is selected from the group consisting of Au, Ag, Pt and indium tin oxide

11. A device according to claim 8 wherein the strength of the electrical potential in chamber 1 is about 100 mV.

12. A device according to claim 1 wherein chamber 2 is configured to achieve electroporation of the T-cells and comprises one or more channels to pattern T-cells.

13. A device according to claim 12 wherein the T-cells are patterned by applying an electrical current to pattern the cells in a single file.

14. The device according to claim 13 wherein the electrical current used to pattern the cells is from about 20 mV to about 500 mV.

15. The device according to claim 14 wherein the electrical current used to pattern the cells is about 100 mV

16. A device according to claim 12 wherein the strength of the DCP current strength in chamber 2 to achieve electroporation is about 1.5V/P.

17. A device according to claim 12 wherein the DCP in chamber 2 is a pulse of from about 1 ms to about 10 ms.

18. A device according to claim 17 wherein the pulse is about 5 ms.

19. A kit comprising the device according to claim 1 combined with one or more of the accessories selected from the group consisting of syringes, nutrients, food, cytokines, buffers, storage containers, transfer vehicles and instructions.

20. The device according to claim 3, wherein the top and bottom surface are parallel to each other and are at a distance of about 100 μm.
